(19) United States
(12) Patent Application Publication
(10) Pub. No.: US 2010/0233128 A1
(43) Pub. Date: Sep. 16, 2010

(54) Natural Skin Products Containing Dairy and Processes

(75) Inventor: Oksana Panasenko, Victoria (CA)

Correspondence Address: VENABLE LLP, P.O. BOX 34385, WASHINGTON, DC, 20043-9998, US

(73) Assignee: MCI MEDIA CAPITAL INC., Victoria (CA)

(21) Appl. No.: 12/722,754

(22) Filed: Mar. 12, 2010

Related U.S. Application Data
(60) Provisional application No. 61/160,009, filed on Mar. 13, 2009.

(30) Foreign Application Priority Data
Mar. 13, 2009 (CA) 2658328
Mar. 27, 2009 (CA) 2660253

(51) Int. Cl. A61K 35/74; A61K 35/20; C11D 3/384; A61Q 19/10

(52) U.S. Cl. 424/93.4; 424/535; 510/158

(57) ABSTRACT

A skincare product containing one or more dairy products, and one or more ingredients such as a natural oil, an herb, a fruit, a vegetable, a grain, honey, sea salt, egg yolk and mixtures thereof, for application to human skin in order to improve the well-being of said skin. The skincare product is applied to the skin in order to cleanse, beautify, promote attractiveness, and/or alter the feel or appearance of the skin. Furthermore, the skincare product is applied to the skin by rubbing, pouring, sprinkling, and/or spraying the skincare product onto the skin.

This application claims priority to U.S. provisional application No. 61/160,009, filed Mar. 13, 2009; and Canadian application nos. 2,658,328, filed Mar. 13, 2009, and 2,660,253, filed Mar. 27, 2009, which are hereby incorporated by reference.

### FIELD OF INVENTION

This invention relates to skincare products containing dairy products and processes used to produce them. The skincare products of the present invention can be used on oily, normal, dry or combination skin. In particular, the invention relates to natural skincare products.

### BACKGROUND

There is a need in the skincare and cosmetic industries to formulate all-natural products that are free from synthetic chemicals or preservatives. Many of these chemicals are quite harsh on the human skin, and actually have negative effects on the health of the skin.

One natural ingredient, milk, in combination with water, is known to have beneficial effects for the skin Milk baths have been common for centuries, comprising milk, buttermilk or dried milk powder mixed with water. It has been observed that the natural properties of milk help to soften and smooth the skin, exfoliate, moisturize, and accelerate skin cell renewal.

In U.S. Pat. No. 4,255,452, Veney discloses a cosmetic cream preparation for cleansing, smoothing and moisturizing the skin basically comprising a mixture with water, non-fat dry milk solids, witch hazel and olive oil.

In U.S. Pat. No. 4,395,424, Veney discloses a cosmetic cream preparation for cleansing, smoothing and moisturizing the skin basically comprising a mixture with water, non-fat dry milk solids, witch hazel, olive oil and a polyoxyethylene having a hydroxyl number of from about 40 to 60. An emollient and preservative may be added and if desirable a fragrance material may also be added. While this composition comprises natural ingredients, such as witch hazel, olive oil, water, and non-fat dry milk solids, it does require the use of a chemical additive (a polyoxyethylene) as a preservative.

Roth teaches in U.S. Pat. No. 4,986,986 a skin moisturizing lotion produced by blending specific quantities of peanut oil, rose water, olive oil, anhydrous lanolin, and natural lemon oil for application to the skin. The product contains no milk, no alcohol, emulsifiers or preservatives. It must be stored at temperatures ranging from about 13° C.-32° C.

French Pat. Publication No. 2 388 555 (Haguenauer) discloses a “nourishing cream for protecting dry skin” which is essentially a lanolin compound, containing the lanolin as its primary and dominant ingredient (60%). The lanolin is treated by heating and mixed with minor amounts of orange juice (other fruit juices may be substituted), almond oil and rose water. While all natural, the cream does not contain any milk.

U.S. Pat. No. 4,297,374 (Wess) discloses a moisturizing and cleansing cream blend of fresh bananas or avocados, baking powder, orange juice and solid or liquid vegetable shortening. While all natural, the cream does not contain any milk.

U.S. Pat. No. 3,810,006 (Sutliff) provides two separate mixtures which are separately applied to the skin and are otherwise never combined. The first mixture combines olive oil and almond oil which is applied to the skin as an oil base and then an aqueous paste is applied over the oil base. The paste contains from 80%-90% milk solids and approximately 20% dried banana flour. While all natural, the mixtures do not contain any milk.

U.S. Pat. No. 3,463,862 (Mazza) discloses a cosmetic lotion using as its essential ingredients albumin, an astringent, and an oil. A typical composition includes proportionate amounts of linseed oil, benzoic acid, egg whites, alum, lanolin, corn oil, glycerin and liquid petrolatum. While all natural, the lotion does not contain any milk.

U.S. Pat. No. 1,631,384 (Richmond) discloses a skin lotion containing pure citrous pectin (e.g., lemon pectin) to which other materials may be added including glycerine, soaps, oils, emulsified oils, lanolin, almond oil, stearic acid, alkali stearates and the like. While all natural, the lotion does not contain any milk.

In addition, U.S. Pat. No. 4,569,839 to Grollier et al. describes cosmetic compositions for the treatment of skin consisting of pulverized particles of at least one plant and a cohesion agent to maintain the homogeneity of the composition. While all natural, the compositions do not contain any milk.

In U.S. Pat. No. 5,431,911, Reynolds discloses a skin treatment composition in the form of a cosmetic paste which comprises almond meal and/or oatmeal, a clay mineral, tincture of benzoin and witch hazel. In an alternate skin treatment composition, a peroxide or lemon juice is substituted for the witch hazel component to provide a composition for the removal of surface blemishes or for bleaching purposes. While all natural, the composition does not contain any milk.

In U.S. Pat. No. 5,139,771, Gerstein discloses a face masque composition useful for treating human skin for improvement comprising about 1-70% of a hydrolyzed grain endproduct, about, 0.1-15% of a seaweed derivative and about 20-95% water. While all natural, the face masque does not contain any milk.

In U.S. Pat. No. 5,660,840, Pruett discloses a therapeutic preparation comprised of bananas, orange juice, and coconut milk, wherein the proportion of the ingredients (by volume) are approximately 90-95% banana, 2-5% orange juice and 2-5% coconut milk. The preparation is in the form of a mask that is to be applied overnight. While all natural, the preparation does not contain any milk.

### SUMMARY OF THE INVENTION

It is desirable to formulate skincare products that exploit the properties of milk and dairy products. It is also desirable to formulate skincare products that are all-natural, i.e., free from synthetic preservatives and chemicals. It is also desirable to maintain freshness of the natural ingredients used in the preparation of these products. These products can be applied topically to the skin. This invention addresses these objectives.

The invention in its general form will first be described, and then its implementation in terms of specific embodiments will be detailed with reference to examples following hereafter. These embodiments are intended to demonstrate the principle of the invention, and the manner of its implementation. The invention in its broadest and more specific forms will then be further described, and defined, in each of the individual claims which conclude this Specification.

### DESCRIPTION OF THE INVENTION

According to embodiments of the present invention, there is provided a skincare product that includes: a) one or more dairy products; and b) one or more ingredients such as a natural oil; an herb; a fruit; a vegetable; a grain; flower-based honey; sea salt; egg yolk; and mixtures thereof, for application to human skin in order to improve the well-being of said skin, wherein the total amount of dairy product is present in an amount of from about 45% to about 85% by volume, from about 50% to about 80% by volume; or from about 60% to about 75% by volume.

Some embodiments of the invention provide a face and body scrub containing, by volume, about 60 parts cottage cheese, about 60 parts sour cream, about 10 parts sesame oil, about 10 parts ground rice, about 1 part lemon oil, about 1 part grapefruit oil, about 1 part bergamot oil, about 1 part lavender oil, about 5 parts olive oil, about 2.5 parts avocado oil, about 1 part rose oil, and about 5 parts apricot oil.

Some embodiments of the invention provide a cream containing, by volume, about 60 parts of blended melon, about 120 parts of fat sour cream; about 30 parts of flower honey; about 5 parts dried peppermint powder; about 2 parts 2 drops peppermint oil, about 1 part bergamot oil; about 1 part myrrh oil; about 1 part ylang ylang oil; about 10 parts avocado oil; about 5 parts olive oil; and about 5 parts sweet almond oil.

Some embodiments of the invention provide a cleanser containing, by volume, about 120 parts sour cream, about 10 parts dried lavender powder, about 10 parts flower honey, about 2.5 parts olive oil, about 1 part rose oil, about 2.5 parts tsp black currant seeds oil, about 2.5 parts lavender oil, about 5 parts hazelnut oil, about 1 part chamomile oil, about 1 part bergamot oil, and about 1 part lemon oil.

Some embodiments of the invention provide a face mask containing, by volume, about 60 parts cottage cheese, about 30 parts sour cream, about 15 parts lemon juice, about 15 parts orange pulp, about 5 parts palmorosa oil, about 1 part bergamot oil, about 1 part neroli oil, about 2.5 parts olive oil and about 2.5 parts avocado oil.

Some embodiments of the invention provide a face mask containing, by volume, about 120 parts sour cream, about 60 parts pumpkin pulp, about 1 part bergamot oil, about 1 part lemon oil, about 1 part grapefruit oil, about 2.5 parts jojobo oil, about 1 part neroli oil, about 2.5 parts olive oil, about 2.5 parts sweet almond oil and about 2.5 parts black currant oil.

Some embodiments of the invention provide a face mask containing, by volume, about 120 parts sour cream, about 10 parts green tea powder, about 10 parts grape seed oil, about 1 part jasmine absolute oil, about 1 part peppermint oil, about 1 part lavender oil, about 1 part chamomile oil, about 2.5 parts olive oil, about 2.5 parts sweet almond oil, about 2.5 parts apricot oil and about 2.5 parts avocado oil.

Some embodiments of the invention provide a face mask containing, by volume, about 60 parts fat cottage cheese, about 60 parts sour cream, about 5 parts dry parsley powder, about 5 parts fish oil, about 2.5 parts lemon peel, about 5 parts sweet almond oil and about 5 parts olive oil.

Some embodiments of the invention provide a face mask containing, by volume, about 30 parts whipping cream, about 120 parts sour cream, about 10 parts of flower honey, about 10 parts dried melissa powder, about 1 part bergamot oil, about 1 part neroli oil, about 1 part chamomile oil and about 1 part rosehip oil.

Some embodiments of the invention provide a face mask containing, by volume, about 120 parts sour cream or about 180 parts skimmed milk; about 60 parts grounded oatmeal, about 30 parts wheat germ oil, about 1 part sandalwood oil and about 1 part rose oil.

Some embodiments of the invention provide a face mask containing, by volume, about 60 parts cottage cheese, about 60 parts sour cream, about 90 parts blended tomatoes (skin removed), about 10 parts sesame oil, about 1 part tea tree oil, about 1 part lavender oil, about 1 part sage oil and about 1 part ylang ylang oil.

Some embodiments of the invention provide a face mask containing, by volume, about 60 parts sour cream, about 60 parts cottage cheese, about 120 parts blended melon, about 1 part tea tree oil, about 1 part chamomile oil, about 1 part bergamot oil, about 2 parts grapefruit oil, about 1 part lavender oil and about 1 part neroli oil.

Some embodiments of the invention provide a face mask containing, by volume, about 120 parts sour cream, about 20 parts parsley juice, about 2 parts bergamot oil, about 1 part grapefruit oil, about 1 part tea tree oil, about 1 part lavender oil, about 1 part chamomile oil, about 2.5 parts olive oil, about 2.5 parts sweet almond oil and about 1 part rose oil.

Some embodiments of the invention provide a face mask containing, by volume, about 120 parts sour cream; about 10 parts dried St John's Wort powder; about 2 parts bergamot oil; about 1 part grapefruit oil; about 1 part tea tree oil; about 1 part lavender oil; about 1 part neroli oil; about 1 part chamomile oil and about 1 part lemon oil. This mask is designed for skin with large pores; it cleanses these pores and shrinks them.

Some embodiments of the invention provide a face mask containing, by volume, about 60 parts sour cream; about 60 parts cottage cheese; about 20 parts carrot juice or pulp; about 40 to 50 parts egg yolk; about 1 part lemon oil; about 1 part chamomile oil; about 1 part mandarin oil; about 2.5 parts olive oil; about 2.5 parts almond oil and about 1 part rose oil. This mask hydrates dry skin.

Some embodiments of the invention provide a face mask containing, by volume, about 90 parts far cottage cheese, about 10 parts avocado oil, about 10 parts jojobo oil, about 2.5 parts olive oil, about 1 part ylang ylang oil, about 2.5 parts rosehip oil, about 1 part neroli oil, about 1 part myrhh oil and about 1 part chamomile oil.

Some embodiments of the invention provide a face mask containing, by volume, about 90 parts sour cream, about 90 parts whipping cream, about 20 parts almond oil, about 30 parts avocado, about 1 part ylang ylang oil, about 1 part myrhh oil, about 1 part chamomile oil, about 1 part neroli oil and about 1 part rose oil.

Some embodiments of the invention provide a face mask containing, by volume, about 60 parts sour cream, about 60 parts whipping cream, about 5 parts of flower honey, about 5 parts of oats, about 30 parts fresh blended raspberries, about 2 parts lavender oil, about 1 part ylang ylang oil and about 1 part chamomile oil.

Some embodiments of the invention provide a face mask containing, by volume, about 60 parts sour cream, about 30 parts cottage cheese, about 20 parts whipping cream, about 10 parts sea salt, about 30 parts sweet almond oil, about 1 part melissa oil and about 1 part sandalwood oil.

Some embodiments of the invention provide a face mask containing, by volume, about 60 parts sour cream; about 60 parts cottage cheese; about 10 parts olive oil; about 10 parts orange peel powder; about 1 part orange essential oil; about 1 part grapefruit essential oil and about 1 part sandalwood oil.

Some embodiments of the invention provide an eye mask containing, by volume, about 60 parts sour cream, about 5 parts dried parsley powder, about 5 parts almond oil, about 5 parts avocado oil and about 5 parts olive oil.

Some embodiments of the invention provide an eye mask containing, by volume, about 120 parts sour cream, about 40 to 50 parts egg yolk; about 5 parts of flower honey, about 10 parts sweet almond oil and about 5 parts avocado oil.

Some embodiments of the invention provide a body scrub containing, by volume, about 120 parts sour cream, about 30 parts almond oil and about 30 parts sea salt.

Some embodiments of the invention provide a body scrub containing, by volume, about 120 parts sour cream, about 10 parts grounded orange peel, about 30 parts blended orange, about 2.5 parts olive oil, about 1 part grapefruit oil, about 1 part lemon oil, about 1 part chamomile oil and about 1 part rose oil.

Some embodiments of the invention provide an all-over bath mask containing, by volume, about 100 parts skimmed milk, 25 parts of flower honey, 6 parts fragrance oil and 6 parts dried lavender flowers.

According to embodiments of the present invention, the skincare product is used to cleanse, beautify, promote attractiveness, and/or alter the appearance of skin. The embodiments of the present invention are useful in that they provide one or more of the following benefits: rejuvenate the skin, reduce wrinkles and fine lines, remove blemishes and blackheads, fight dark spots on the skin, remove make-up, clear pores, reduce harmful effects of pollution, increase facial collagen and control oily skin. These products can also be directed to the reduction of puffiness and dark circles around the eyes. In addition, some embodiments, such as scrubs, are used to slough dead skin cells on various parts of the body; to improve microcirculation; and to reduce cellulite.

The skincare product of the present invention can be used on oily, normal, dry or combination skin. In embodiments, the skincare product is rubbed, poured, sprinkled, and/or sprayed onto the skin. In some embodiments, the skincare product is free from all synthetic additives and preservatives.

“Dairy product” refers to foodstuff produced from milk. The term milk is also used for whitish non-animal substitutes such as soy milk, rice milk, almond milk, and coconut milk. The milk obtained from animals can be pasteurized or unpasteurized; homogenized or non-homogenized. In addition, the milk can be animal-based milk obtained from, but not limited to animals such as cows, buffalo, camels, donkeys, goats, horses, reindeer, sheep, water buffalo, yaks and moose. Animal-based milk can also be organic, in that the milk is produced without the use of chemical herbicides or pesticides, and generally with more natural fertilizers and higher standards for the animals. The milk can also be “Demeter certified”, in that it is produced with biodynamic agriculture methods and is similar in standards to organic milk.

According to some embodiments of the invention, the dairy products used in the present invention can also be based on milk to which are added bacterial cultures. Examples of bacterial cultures added to milk are not limited to, but include Lactobacillus acidophilus (“acidophilus milk”), bifidobacteria (“a/B milk”) and Lactococcus lactis (“cultured buttermilk”). Both acidophilus milk and a/B milk are consumed by lactose-intolerant individuals. In exemplary skincare products, the dairy product is fermented after addition of bacterial cultures.

Natural skincare products that are free from all synthetic additives and preservatives are sensitive to microbial contamination. The addition of bacterial cultures results in lactic acid fermentation of milk. The lactic acid creates a low pH environment that can help inhibit microbial growth. The addition of bacterial cultures to milk therefore provides a natural way of helping preserve the skincare products of the present invention.

The addition of bacterial cultures to milk is also advantageous because the product of bacterial fermentation, i.e., lactic acid, can be beneficial to the skin. Lactic acid acts as an exfoliant, removing dead cells from the skin and enhances the overall appearance of the skin. Exfoliated skin will have a healthier glow, reduced fine lines and wrinkles, and a smoother appearance.

Accordingly, embodiments of the present invention include skincare products having a pH of less than about 7.0. In some embodiments, the pH of the skincare product is less than about 5.0, and in exemplary embodiments, the pH is less than about 4.6, less than about 4.0, or less than about 3.8. In embodiments, the skincare product has a pH from about 3.0 to 5.0. In other embodiments, the skincare product has a pH from about 3.4 to 3.8. In still further embodiments, the skincare product has a pH from about 3.6 to 3.8.

In exemplary embodiments of the invention, the skincare product is prepared from a fermented dairy product having a pH of less than about 7.0. In some exemplary embodiments, the pH of the fermented dairy product is less than about 5.0, less than about 4.6, less than about 4.0, or less than about 3.8. In embodiments, the fermented dairy product has a pH from about 3.0 to 5.0. In other embodiments, the fermented dairy product has a pH from about 3.4 to 3.8. In still further embodiments, the fermented dairy product has a pH from about 3.6 to 3.8.

In addition, the dairy products used to produce the skincare products of the present invention can be based on commercially sold milk, which commonly has vitamin D added to it. In addition, reduced fat milks can have added vitamin A.

Pasteurization is used to kill harmful microorganisms by heating the milk for a short time and then cooling it for storage and transportation. Pasteurized milk is and must be stored in a cold environment. Skincare products of the present invention based on dairy products produced from pasteurized or non-pasteurized milk are, and must be stored at temperatures from about 1° C. to about 4° C., or frozen at a temperature of about −18° C. (0° F.) for maximum efficiency and safety. In addition, these skincare products can have a shelf life of between 7-60 days, depending on the type of dairy product used.

On the other hand, ultra pasteurization or ultra-high temperature treatment (UHT), heats the milk to a higher temperature for a shorter time. This extends its shelf life and allows the unopened milk to be stored unrefrigerated because of the longer lasting sterilization effect. As such, skincare products of the present invention that comprise UHT-based dairy products have an extended shelf-life. Furthermore, skincare products of the present invention that are prepared under sterile conditions and comprise UHT-based dairy products do not require refrigeration as long as sterility is preserved.

Nonlimiting examples of dairy products used in the present invention include milk (with varying fat content); cream; crème fraîche; smetana, sour cream, whipping cream, clotted cream cultured buttermilk, kefir (fermented cultured milk drink resembling buttermilk but based on different yeast and bacteria culture); buttermilk; skim milk; condensed milk; evaporated milk; ricotta cheese, “khoa” (milk heated and reduced in volume); baked milk, butter; buttermilk, ghee, cheese; curds; cottage cheese; quark; cream cheese, fromage frais, casein (including caseinates; milk protein concentrates and isonates; whey protein concentrates and isonates; hydrolysates; and mineral concentrates), yoghurt (including ayran and lassi), whey, gelato, ice cream (including ice milk, frozen custard and frozen yoghurt), kumis/airag, viili, kajmak, kefir, filmjölk, piimä, vla, dulce de leche and uloo kao patha laeen ka.

The dairy products used in the production of the skincare products of the present invention have all variations of fat content (e.g., 0%, low-fat, 7%, etc.), and can be based on soy milk, rice milk, coconut milk or almond milk and milk product substitutes, such as tofu and other vegan substitutes.

The dairy products can be non-pasteurized or pasteurized by the regular process, and are suitably selected from skim milk, cottage cheese, sour cream, whipping cream and mixtures thereof.

In some embodiments of the present invention, the skincare product is packaged for single use. Single use packages preserves the sterility of the product and conveniently provides the user a pre-measured amount of product. Single use packages are well known to one of skill in the art. A non-limiting example of a single use package is a container with a removable lid. Materials for making the container and lid are well known in the art. For example, the container and lid can be made from, but is not limited to, a plastic, including a thermoplastic. In addition, the container and lid can be made from the same or from different materials. Another non-limiting example of a single use package is a pouch with a front wall, a back wall, and hermetically sealed sides. Optionally, an applicator wand is stored in the pouch. The single use package can also be a rigid container with an opening for an applicator wand. The rigid container has a reservoir for the skincare product. A further non-limiting example of a single use package is a squeezable tubular body. The application of pressure, e.g., squeezing, to the tubular body facilitates expulsion of the product from the product reservoir.

Accordingly, embodiments of the present invention include a single-use package comprising the claimed skincare product. In some embodiments, the single-use package contains between 1 to 3 ounces of skincare product. In other embodiments, the single-use package contains 2 ounces of skincare product.

Nonlimiting examples of natural oils used in the present invention include basic oils such as almond oil, apricot kernel, avocado, borage, carrot oil, castor oil, evening primrose, grapeseed, hazelnut, hemp oil, jojobo, neem oil, peanut, rose hip seed, black current seed oil, sesame seed oil, vegetable glycerine, Vitamin E, rise bran oil, macadamia oil, emu oil, coconut oil virgin, walnut oil, corn oil, flaxseed oil, olive oil, palm oil, canola oil and safflower seed oil; and essential oils such as agar oil, ajwain oil, angelica root oil, anise oil, asafoetida, balsam oil, basil oil, benzoin, bergamot oil, black pepper oil, buchu oil, cajeput, camphor, cannabis flower oil, caraway oil, cardamom seed oil, carrot seed oil, cedarwood oil, chamomile, calamus root, cinnamon oil, cistus species, citronella oil, cinnamon leaf, clary sage, clove leaf oil, coffee, costmary oil (bible leaf oil), costus root, cranberry seed oil, cubeb, cumin oil/black seed oil, cypress, cypriol, curry leaf, davana oil, dill oil, elecampane, eucalyptus oil, fennel seed oil, fenugreek oil, fir, fish oil, frankincense oil, galangal, galbanum, geranium oil, helichrysum, hyssop, hyacinth, idaho tansy, jasmine oil, jasmine absolute oil, juniper berry oil, kanuka, laurus nobilis, lavender oil, ledum, lemon oil, lemongrass, lime, litsea cubeba oil, mandarin (red), marjoram, melaleuca see tea tree oil, melissa oil (lemon balm), mentha arvensis oil/mint oil, mountain savory, mugwort oil, mustard oil, myrrh oil, myrtle, neroli, nutmeg, orange oil, oregano oil, orris oil, palo santo, parsley oil, patchouli oil, perilla essential oil, peppermint oil, petitgrain, pine oil, ravensara, red cedar, roman chamomile, rose oil, rosehip oil, rosemary oil, rosewood oil, sage oil, sandalwood oil, sassafras oil, savory oil, schisandra oil, spearmint oil, spikenard, spruce, star anise oil, sweet birch, tagetes, tangerine, tarragon oil, tea tree oil, thyme oil, tsuga, turmeric, valerian, vetiver oil (khus oil), western red cedar, wheat germ oil, wintergreen, yarrow oil, ylang-ylang and zedoary. Oils can be obtained from plants, including whole plants or plant parts. Plant parts include stems, leaves, bark, flowers, fruits, and roots. Oils are obtained using methods known in the art.

Exemplary oils are selected from sesame oil, lemon oil, grapefruit oil, bergamot oil, lavender oil, olive oil, avocado oil, sweet almond oil, rose oil, apricot oil, myrrh oil, peppermint oil, ylang ylang oil, black currant seeds oil, hazelnut oil, chamomile oil, palmorosa oil, neroli oil, jojobo oil, black currant oil grape seed oil, jasmine absolute oil, fish oil, rosehip oil, wheat germ oil, sandalwood oil, sage oil, tea tree oil, Melissa oil, orange essential oil, grapefruit essential oil, almond oil, sweet almond oil and mixtures thereof.

Herbs used in the present invention can be fresh or dried; these include, for example: angelica, anise hyssop, anise, Artemisia, sweet basil, bee balm, borage, burnet, calendula (pot marigold), caraway, catnip, chamomile (sweet false), chamomile, chervil, chives, cilantro, clary sage, coriander, costmary, dill, fennel, feverfew, geranium, germander, green tea, horehound, hyssop, lavender, lemon verbena, lemon balm, lovage, marjoram, melissa, nasturtium, oregano, parsley, peppermint, perilla, rosemary, rue, sage (pineapple) santolina, sorrel, southernwood, spearmint, summer savory, St. John's Wort, sweet woodruff, sweet rocket, sweet marjoram, tansy, tarragon, thyme, valerian, winter savory, wormwood, tea and yarrow.

In exemplary embodiments, herbs used in the preparation of the skincare product of the present invention are selected from dried peppermint powder, dried lavender powder, green tea powder, dry parsley powder, parsley juice, Melissa powder, St. John's Wort powder, lavender flowers and mixtures thereof.

Fruits used in the present invention can be fresh, dried, pulped or juiced; these include, for example: bananas (all variations), apple (all variations), fig, pear (all variations), apricot, nectarine, plum, cherry (all variations), peach, quince, clementine, grapefruit, kumquat, lemon, lime, mandarin, minneola, orange, pummelo, satsuma, sweety, tangerine, tangelo, ugli, akee, araza, durian, jaboticaba, kepel, ketupa, loquat, lucuma, mangosteen, persimmon, rambai, sala, salak, santoli, kechapi, sapodilla, voavanga, alibertia, bael fruit, avocado, bignay, date, gandaria, golden apple, hogplum, indian jujube, ita palm, mango, lychees, mammey sapote, nance, otaheite, gooseberry (all variations), peach palm, rambutan, sansapote, star nut palm, sea grape, velvet tamarind, surinam cherry, biriba, breadfruit, champedak, mountain soursop, marinda, nangka, jackfruit, pineapple, soncoya, sweetsop olives (all variations), guava, kiwano, kiwi fruit, monkey bread, passion fruit, papaya, philippine roseapple, pitahaya, pomegranate, sweet granadilla, tomato (all variations), chalta bilberry, blackberry, blueberry, boysenberry, cranberry, currants (red and white), grapes, raspberry, huckleberry, strawberry, canary melon, cantaloupe, casaba, crenshaw, charantais, christmas melon, derishi, galia, horned melon/kiwano, melon-pear, musk melon, net melon, ogen melon, pepino melon, persian melon, russian/uzbek melon, sharlyn, sweet melon, tree melon, watermelon (all variations), wax melon, winter melon, honeydew, santa claus, xigua, cannonball, feijoa, genipap, giant granadilla and calabash. In addition, different portions of the fruit (such as the peel, seeds, etc) can be used in various embodiments of the present invention.

Fruits used in the preparation of skincare products according to exemplary embodiments of the present invention include melon, lemon, orange, avocado, raspberry and mixtures thereof. In particular, orange pulp, orange peel, lemon juice and lemon peel are preferred forms of orange and lemon used.

Vegetables used in the present invention can be fresh, pulped or juiced, and include, but are not limited to, artichoke, asparagus, aubergine, beans (all variations), beet, broccoli, brussels sprouts, cabbage, carrot, cauliflower, celeriac, celery, chard, chicory, collards, corn, cress, cucumbers, eggplant, gourds (members include cucumbers, squashes, luffas, and melons), jerusalem artichoke, kales, kohlrabi, leek, lettuce, melons, mushrooms, okra, onions, parsnips, peas, peppers, potatoes, pumpkins, radicchio, radish, rhubarb, rutabaga, shallots, spinach, squash, swede, sweetcorn, sweet potato, turnips, watercress, yams and zucchini. The vegetable may be in raw, cooked or dried form.

Exemplary vegetables used in preparing skincare products according to the present invention include pumpkin, tomatoes and carrot.

In some embodiments, grains are grounded. In exemplary embodiments, the grains are rice, oats, barley, wheat, flax, corn and mixtures thereof. In still further embodiments, the corn is in the form of cornmeal; the oat is in the form of oatmeal, and the flour is a mixture of grains.

Other optional nutrients used in the preparation of the skincare product of the present invention include honey (from flowers), sea salt and egg yolk.

Embodiments of the present invention include, but are not limited to, face masks, eye masks, crèmes, body scrubs, facial scrubs, cleansers and all-over body masks.

The skincare products of the present invention can include from about 45% to about 85% by volume of dairy product, and from about 15% to about 55% by volume of other natural ingredients. Exemplary embodiments include from about 50% to about 80% by volume of dairy product, from about 20% to about 50% by volume of other natural ingredients; from about 60% to about 75% by volume of dairy product, and from about 25% to about 40% by volume of other natural ingredients. Artificial dyes and perfumes are excluded from the composition in exemplary embodiments.

In embodiments, the compositions of the present invention are made as follows: a) one or more dairy products are blended together; b) basic and/or essentials oils are combined together; c) herbs, vegetables, fruit, grains and/or other ingredients are chopped/mixed to a required solution consistency; d) after all of the separate ingredients are prepared, they are combined together to an even consistency and e) the final composition is stored at temperatures between from about 1° C. to about 4° C., or frozen at a temperature of about −18° C. (0° F.) for maximum efficiency and safety.

In embodiments, the compositions of the present invention are used to cleanse, beautify, promote attractiveness, and/or alter the appearance of skin. In further embodiments, the compositions of the present invention are rubbed, poured, sprinkled, and/or sprayed onto the skin.

The foregoing summarizes the principal features of the invention and some of its optional aspects. The invention may be further understood by the description of the preferred embodiments, in conjunction with the drawings, which now follow.

Wherever ranges of values are referenced within this specification, sub-ranges therein are intended to be included within the scope of the invention unless otherwise indicated. Where characteristics are attributed to one or another variant of the invention, unless otherwise indicated, such characteristics are intended to apply to all other variants of the invention where such characteristics are appropriate or compatible with such other variants.

### EXAMPLES

In all of the examples below, 1 drop has a volume range of from about 0.3 ml to about 0.5 ml; 1 teaspoon has a volume range of about 3 ml to about 6 ml; and 1 tablespoon has a volume range of about 13 ml to about 17 ml.

### Example 1

A face and body scrub of the present invention consists of 2 tbsp cottage cheese, 2 tbsp sour cream, 1 tsp sesame oil, 1 tsp ground rice, 1 drop lemon oil, 1 drop grapefruit oil, 1 drop bergamot oil, 1 drop lavender oil, ½ tsp olive oil, ¼ tsp avocado oil, 1 drop rose oil, and ½ tsp apricot oil. This face-and-body scrub opens pores and increases the skin's oxygen intake while it gently cleans away and removes dead skin and oil.

### Example 2

A crème of the present invention consists of: 2 tbsp of blended melon, 4 tbsp fat sour cream; 1 tbsp flower honey; ½ tsp dried peppermint powder; 2 drops peppermint oil 1 drop bergamot oil; 1 drop myrrh oil; 1 drop ylang ylang oil; 1 tsp avocado oil; ½ tsp olive oil; ½ tsp sweet almond oil. This crème relaxes, refreshes and reduces the harmful effects of airborne pollution on the skin.

### Example 3

A cleanser of the present invention consists of: 4 tbsp sour cream, 1 tsp dried lavender powder, 1 tsp flower honey, ¼ tsp olive oil, 1 drop rose oil, ¼ tsp black currant seeds oil, 5 drops lavender oil, ½ tsp hazelnut oil, 1 drop chamomile oil, 1 drop bergamot oil, 1 drop lemon oil. This cleanser can be used for all types of skins and is especially effective in controlling oily skin. This cleanser provides natural anti-bacterial agents; it cleanses and nourishes the skin to improve microcirculation which, in turn, reduces imitation and inflammation.

### Example 4

A face mask of the present invention consists of 2 tbsp cottage cheese, 1 tbsp sour cream ½ tbsp lemon juice, ½ tbsp orange pulp, ½ tsp palmorosa oil, 1 drop bergamot oil, 1 drop neroli oil, ¼ tsp olive oil and ¼ tsp avocado oil. This face mask is applicable for all skin types, reduces wrinkles, revitalizes the skin and replaces lost nutrients.

### Example 5

A face mask of the present invention consists of 4 tbsp sour cream, 2 tbsp pumpkin pulp, 1 drop bergamot oil, 1 drop lemon oil, 1 drop grapefruit oil, ¼ tsp jojobo oil, 1 drop neroli oil, ¼ tsp olive oil, ¼ tsp sweet almond oil and ¼ tsp black currant oil. This face mask assists in combating dark spots on the skin.

### Example 6

A face mask of the present invention consists of 4 tbsp sour cream, 1 tsp green tea powder, 1 tsp grape seed oil, 1 drop jasmine absolute oil, 1 drop peppermint oil, 1 drop lavender oil, 1 drop chamomile oil, ¼ tsp olive oil, ¼ tsp sweet almond oil, ¼ tsp apricot oil and ¼ tsp avocado oil. This mask contains green tea which is an antioxidant. As a face mask, it helps increase facial collagen.

### Example 7

A face mask of the present invention consists of 2 tbsp fat cottage cheese, 2 tbsp sour cream, ½ tsp dry parsley powder, ½ tsp fish oil, ¼ tsp lemon peel, ½ tsp sweet almond oil and ½ tsp olive oil. This mask moisturizes the skin and provides vitamins to help restore its protective layer.

### Example 8

A face mask of the present invention consists of 3 tsp of whipping cream, 4 tbsp sour cream, 1 tsp of flower honey, 1 tsp dried melissa powder, 1 drop bergamot oil, 1 drop neroli oil, 1 drop chamomile oil and 1 tsp rosehip oil. This mask contains Melissa, which has been found to be an excellent antioxidant. This mask helps tone the skin and tighten pores when used on a regular basis.

### Example 9

An eye-and-face mask of the present invention consists of: 4 tbsp sour cream or 6 tbsp skimmed milk, 2 tbsp grounded oatmeal, 1 tbsp wheat germ oil, 1 drop sandalwood oil and 1 drop rose oil. This morning eye-and-face-mask tightens pores and eliminates blackheads. This mask can also be used for removing makeup.

### Example 10

A face mask of the present invention consists of 2 tbsp cottage cheese, 2 tbsp sour cream, 3 tbsp blended tomatoes (skin removed), 1 tsp sesame oil, 1 drop tea tree oil, 1 drop lavender oil, 1 drop sage oil and 1 drop ylang ylang oil. This mask is designed for oily skin; it removes excess oil, softens and cleanses the skin of impurities.

### Example 11

A face mask of the present invention consists of 2 tbsp sour cream, 2 tbsp cottage cheese, 4 tbsp blended melon, 1 drop tea tree oil, 1 drop chamomile oil, 1 drop bergamot oil, 2 drops grapefruit oil, 1 drop lavender oil and 1 drop neroli oil. This mask is designed for lightening the skin.

### Example 12

A face mask of the present invention consists of 4 tbsp sour cream, 2 tsp parsley juice, 2 drops bergamot oil, 1 drop grapefruit oil, 1 drop tea tree oil, 1 drop lavender oil, 1 drop chamomile oil, ¼ tsp olive oil, ¼ tsp sweet almond oil and 1 drop rose oil. This mask is designed for oily skin; it cleanses the pores while making the skin lighter and evening the face's natural color.

### Example 13

A face mask of the present invention consists of 4 tbsp sour cream; 1 tsp dried St John's Wort powder; 2 drops bergamot oil; 1 drop grapefruit oil; 1 drop tea tree oil; 1 drop lavender oil; 1 drop neroli oil; 1 drop chamomile oil and 1 drop lemon oil. This mask is designed for skin with large pores; it cleanses these pores and shrinks them.

### Example 14

A face mask of the present invention consists of 2 tbsp sour cream; 2 tbsp cottage cheese; 2 tsp carrot juice or pulp; 1 egg yolk (about 20-25 ml of egg yolk); 1 drop lemon oil; 1 drop chamomile oil; 1 drop mandarin oil; ¼ tsp olive oil; ¼ tsp almond oil and 1 drop rose oil. This mask hydrates dry skin.

### Example 15

A face mask of the present invention consists of 3 tbsp far cottage cheese, 1 tsp avocado oil, 1 tsp jojobo oil, ¼ tsp olive oil, 1 drop ylang ylang oil, ¼ tsp rosehip oil, 1 drop neroli oil, 1 drop myrhh oil and 1 drop chamomile oil. This mask moisturizes the skin to reduce micro wrinkles and tiny skin cracks.

### Example 16

A face mask of the present invention consists of 3 tbsp sour cream, 3 tbsp whipping cream, 2 tsp almond oil, 1 tbsp avocado, 1 drop ylang ylang oil, 1 drop myrhh oil, 1 drop chamomile oil, 1 drop neroli oil and 1 drop rose oil. This face mask moisturizes dry skin and restores it to its natural, healthy state.

### Example 17

A face mask of the present invention consists of 2 tbsp sour cream, 2 tbsp whipping cream, ½ tsp of flower honey, ½ tsp of oats, 3 tsp fresh blended raspberries, 2 drops lavender oil, 1 drop ylang ylang oil and 1 drop chamomile oil. This face mask is designed to shelter the skin from the sun's harmful ray, in addition to soothing and moisturizing sunburned skin while protecting against further damage.

### Example 18

A face mask of the present invention consists of 2 tbsp sour cream, 1 tbsp cottage cheese, 2 tsp whipping cream, 1 tsp sea salt, 1 tbsp sweat almond oil, 1 drop melissa oil and 1 drop sandalwood oil. This face mask moistens, rejuvenates and brightens dry or weathered skin.

### Example 19

A face mask of the present invention consists of 2 tbsp sour cream; 2 tbsp cottage cheese; 1 tsp olive oil; 1 tsp orange peel powder; 1 drop orange essential oil; 1 drop grapefruit essential oil and 1 drop sandalwood oil. This mask is designed for combination skin; it freshens, moisturizes and reduces fine wrinkles.

### Example 20

An eye mask of the present invention consists of 2 tbsp sour cream, ½ tsp dried parsley powder, ½ tsp almond oil, ½ tsp avocado oil and ½ tsp olive oil. This eye mask is used to reduce fine lines around the eyes.

### Example 21

An eye mask of the present invention consists of 4 tbsp sour cream, 1 egg yolk (about 20-25 ml of egg yolk); ½ tsp of flower honey, 1 tsp sweet almond oil and ½ tsp avocado oil. This eye mask is designed to reduce puffiness around the eyes, and provide overall rejuvenation around the eyes.

### Example 22

A body scrub of the present invention consists of 4 tbsp sour cream, 1 tbsp almond oil and 1 tbsp sea salt. This total body scrub improves microcirculation, reduces cellulite and rejuvenates the skin. It is applicable to areas such as knees, elbows and heels.

### Example 23

A body scrub of the present invention consists of 4 tbsp sour cream, 1 tsp grounded orange peel, 1 tbsp blended orange, ¼ tsp olive oil, 1 drop grapefruit oil, 1 drop lemon oil, 1 drop chamomile oil and 1 drop rose oil. This scrub replenishes the skin with vitamins, reduces cellulite, and removes dead skin cells.

### Example 24

An all-over bath mask of the present invention consists of 1 litre of skimmed milk, 250 ml of flower honey, 4 tbsp fragrance oil and 4 tbsp dried lavender flowers. This bath mask moisturizes, nourishes and revitalizes the skin.

### CONCLUSION

The foregoing has constituted a description of specific embodiments showing how the invention may be applied and put into use. These embodiments are only exemplary. The invention in its broadest, and more specific aspects, is further described and defined in the claims which now follow.

These claims, and the language used therein, are to be understood in terms of the variants of the invention which have been described. They are not to be restricted to such variants, but are to be read as covering the full scope of the invention as is implicit within the invention and the disclosure that has been provided herein.

What is claimed is:

1. A skincare product comprising: a) one or more dairy products; and b) one or more ingredients selected from the groups consisting of a natural oil, an herb, a fruit, a vegetable, a grain, honey, sea salt, egg yolk and mixtures thereof, for application to human skin in order to improve the well-being of said skin, wherein the total amount of dairy product is present in an amount of from about 45% to about 85% by volume.

2. The skincare product of claim 1, wherein the skincare product is stored at a temperature of from about 1° C. to about 4° C.

3. The skincare product of claim 1, wherein the skincare product is frozen at a temperature of about −18° C. (0° F.).

4. The skincare product of claim 1, wherein the skincare product is a face mask, an eye mask, a cream, a body scrub, a facial scrub, a cleanser or a body mask.

5. The skincare product of claim 1, wherein the skincare product has a pH less than about 5.0.

6. The skincare product of claim 5, wherein the skincare product has a pH less than about 4.0.

7. The skincare product of claim 1 comprising from about 50% to about 80% by volume of dairy product.

8. The skincare product of claim 1, wherein the dairy product is selected from the group consisting of animal milk, soy milk, rice milk, almond milk, coconut milk, milk with varying fat content, cream, crème fraîche, smetana, sour cream, whipping cream, clotted cream, cultured buttermilk, kefir, buttermilk, skim milk, condensed milk, evaporated milk, ricotta cheese, khoa, baked milk, butter, buttermilk, ghee, cheese, curds, cottage cheese, quark, cream cheese, fromage frais, casein, milk protein concentrates and isonates, whey protein concentrates and isonates, hydrolysates, yoghurt whey, gelato, ice cream, ice milk, frozen custard, frozen yoghurt, kumis/airag, viili, kajmak, filmjölk, piimä, vla, dulce de leche, uloo kao patha laeen ka and mixtures thereof.

9. The skincare product of claim 8, wherein the dairy products is selected from the group consisting of skim milk, soy milk, cottage cheese, sour cream, whipping cream, yoghurt, cultured buttermilk, acidophilus milk, a/B milk and mixtures thereof.

10. The skincare product of claim 1, wherein the oil is selected from the group consisting of basic oils such as almond oil, apricot kernel, avocado, borage, carrot oil, castor oil, evening primrose, grapeseed, hazelnut, hemp oil, jojobo, neem oil, peanut, rose hip seed, black current seed oil, sesame seed oil, vegetable glycerine, Vitamin E, rise bran oil, macadamia oil, emu oil, coconut oil virgin, walnut oil, corn oil, flaxseed oil, olive oil, palm oil, canola oil and safflower seed oil; and essential oils such as agar oil, ajwain oil, angelica root oil, anise oil, asafoetida, balsam oil, basil oil, benzoin, bergamot oil, black pepper oil, buchu oil, cajeput, camphor, cannabis flower oil, caraway oil, cardamom seed oil, carrot seed oil, cedarwood oil, chamomile, calamus root, cinnamon oil, cistus species, citronella oil, cinnamon leaf, clary sage, clove leaf oil, coffee, costmary oil (bible leaf oil), costus root, cranberry seed oil, cubeb, cumin oil/black seed oil, cypress, cypriol, curry leaf, davana oil, dill oil, elecampane, eucalyptus oil, fennel seed oil, fenugreek oil, fir, fish oil, frankincense oil, galangal, galbanum, geranium oil, helichrysum, hyssop, hyacinth, idaho tansy, jasmine oil, jasmine absolute oil, juniper berry oil, kanuka, laurus nobilis, lavender oil, ledum, lemon oil, lemongrass, lime, litsea cubeba oil, mandarin (red), marjoram, melaleuca see tea tree oil, melissa oil (lemon balm), mentha arvensis oil/mint oil, mountain savory, mugwort oil, mustard oil, myrrh oil, myrtle, neroli, nutmeg, orange oil, oregano oil, orris oil, palo santo, parsley oil, patchouli oil, perilla essential oil, peppermint oil, petitgrain, pine oil, ravensara, red cedar, roman chamomile, rose oil, rosehip oil, rosemary oil, rosewood oil, sage oil, sandalwood oil, sassafras oil, savory oil, schisandra oil, spearmint oil, spikenard, spruce, star anise oil, sweet birch, tagetes, tangerine, tarragon oil, tea tree oil, thyme oil, tsuga, turmeric, valerian, vetiver oil (khus oil), western red cedar, wheat germ oil, wintergreen, yarrow oil, ylang-ylang, zedoary and mixtures thereof.

11. The skincare product of claim 1, wherein the herb is used fresh or dried, and is selected from the group consisting of angelica, anise hyssop, anise, Artemisia, sweet basil, bee balm, borage, burnet, calendula, caraway, catnip, chamomile, chamomile, chervil, chives, cilantro, clary sage, coriander, costmary, dill, fennel, feverfew, geranium, germander, green tea, horehound, hyssop, lavender, lemon verbena, lemon balm, lovage, marjoram, melissa, nasturtium, oregano, parsley, peppermint, perilla, rosemary, rue, sage, santolina, sorrel, southernwood, spearmint, summer savory, St. John's Wort, sweet woodruff, sweet rocket, sweet marjoram, tansy, tarragon, thyme, valerian, winter savory, wormwood, tea, yarrow and mixtures thereof.

12. The skincare product of claim 1, wherein the fruit is fresh, dried, pulped, or juiced; and is selected from the group consisting of banana, apple, fig, pear, apricot, nectarine, plum, cherry, peach, quince, clementine, grapefruit, kumquat, lemon, lime, mandarin, minneola, orange, pummelo, satsuma, sweety, tangerine, tangelo, ugli, akee, araza, durian, jaboticaba, kepel, ketupa, loquat, lucuma, mangosteen, persimmon, rambai, sala, salak, santoli, kechapi, sapodilla, voavanga, alibertia, bael fruit, avocado, bignay, date, gandaria, golden apple, hogplum, indian jujube, ita palm, mango, lychees, mammey sapote, nance, otaheite, gooseberry, peach palm, rambutan, sansapote, star nut palm, sea grape, velvet tamarind, surinam cherry, biriba, breadfruit, champedak, mountain soursop, marinda, nangka, jackfruit, pineapple, soncoya, sweetsop, olive, guava, kiwano, kiwi fruit, monkey bread, passion fruit, papaya, philippine roseapple, pitahaya, pomegranate, sweet granadilla, tomato, chalta, bilberry, blackberry, blueberry, boysenberry, cranberry, red currant, white currant, grape, raspberry, huckleberry, strawberry, canary melon, cantaloupe, casaba, crenshaw, charantais, christmas melon, derishi, galia, horned melon/kiwano, melon-pear, musk melon, net melon, ogen melon, pepino melon, persian melon, russian/uzbek melon, sharlyn, sweet melon, tree melon, watermelon, wax melon, winter melon, honeydew, santa claus, xigua, cannonball, feijoa, genipap, giant granadilla, calabash and mixtures thereof.

13. The skincare product of claim 1, wherein the vegetable is fresh, pulped or juiced, and selected from the group consisting artichoke, asparagus, aubergine, beans (all variations), beet, broccoli, brussels sprouts, cabbage, carrot, cauliflower, celeriac, celery, chard, chicory, collards, corn, cress, cucumbers, eggplant, gourds, jerusalem artichoke, kales, kohlrabi, leek, lettuce, melons, mushrooms, okra, onions, parsnips, peas, peppers, potatoes, pumpkins, radicchio, radish, rhubarb, rutabaga, shallots, spinach, squash, swede, sweetcorn, sweet potato, turnips, watercress, yams, zucchini and mixtures thereof.

14. The skincare product of claim 1, wherein the grain is ground and is selected from the group consisting of rice, oats, barley, wheat, flax, corn and mixtures thereof.

15. A single use package comprising the skincare product of claim 1, and optionally an applicator wand.

16. The single use package of claim 15, wherein the single use package comprises from about 1 to about 3 ounces of the skincare product of claim 1.

17. A kit comprising: (i) a single use package comprising the skincare product of claim 1, and (ii) optionally an applicator wand.

18. The skincare product of claim 1, wherein at least one dairy product comprises bacterial cultures.

19. The skincare product of claim 18, wherein the dairy product comprising bacterial cultures is fermented.

20. A method of cleansing, beautifying, promoting attractiveness, or altering the feel or appearance of skin comprising applying a skincare product according to claim 1 to the skin.

21. The method of claim 20, wherein applying comprises one or more of rubbing, pouring, sprinkling, or spraying the skincare product onto the skin.
